(19) United States
(12) Patent Application Publication
(10) Pub. No.: US 2005/0143748 A1
(43) Pub. Date: Jun. 30, 2005

(54) Pressurizer

(76) Inventors: Carlos Negroni, San Juan, PR (US); Bruce D. Henniges, Galesburg, MI (US); Christopher M. Tague, Delton, MI (US)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 10/988,235

(22) Filed: Nov. 12, 2004

Related U.S. Application Data
(60) Provisional application No. 60/520,232, filed on Nov. 14, 2003.

(51) Int. Cl. A61F 2/28

(52) U.S. Cl. 606/94

(57) ABSTRACT

A pressurizer is provided for use with a bone cement injection device to inject bone cement into a bone canal under pressure. The pressurizer includes a body having proximal and distal ends with an inlet defined at the proximal end, an outlet defined at the distal end, and a channel defined between the inlet and the outlet. The channel includes first and second segments. The first segment extends from the inlet about a first axis to the second segment. The second segment extends from the first segment to the outlet about a second axis intersecting the first axis at an acute angle. The channel defines a cement flow path that directionally varies between the first and second segments such that the bone cement injection device can be angled relative to the bone canal. Alternative embodiments in which the pressurizer includes a rigid support member and a handle are also provided.

### RELATED APPLICATIONS

This application claims the benefit of U.S. provisional patent application Ser. No. 60/520,232, filed Nov. 14, 2003, the advantages and disclosure of which are herein incorporated by reference.

### FIELD OF THE INVENTION

The present invention generally relates to a pressurizer for use with a bone cement injection device to inject bone cement into a bone canal or other anatomical site under pressure.

### BACKGROUND OF THE INVENTION

Total hip arthroplasty (THA) is a procedure that relieves pain and increases mobility of patients having discomfort and partial immobility caused by degenerated natural hip joints. In the THA procedure, an artificial hip joint replaces the natural hip joint. In particular, a spherical end of the patient's femur is removed and replaced with a metallic implant. The metallic implant comprises a stem and a ball fixed to the stem. The stem fits down the center of the femur in a surgically prepared bone canal, i.e., a medullary canal, and is fixed in place by bone cement. The bone cement provides a rigid structure to lock the stem of the metallic implant in the medullary canal. The ball of the metallic implant fits into a socket of an acetabular component to complete the artificial hip joint.

Several factors affect the success of the THA procedure with respect to the preparation and injection of the bone cement into the medullary canal. One of the most important factors is the pressure at which the bone cement is supplied into the medullary canal. Typically, the bone cement is injected into the medullary canal through a nozzle of a bone cement injection device. Once the medullary canal is filled with bone cement, the metallic implant is inserted into the bone cement. The bone cement hardens between the bone and the metallic implant. Fixation of the metallic implant relies upon the interlocking of the bone cement between cancellous bone, which forms an inner surface of the medullary canal, and keyed features or projections on the metallic implant.

In the THA procedure, problems can occur with inadequate penetration of the bone cement into the cancellous bone of the inner surface. As a result, the metallic implant loosens prematurely, resulting in failure of the artificial hip joint. This penetration can be improved by pressurizing the bone cement within the medullary canal. Pressurization can be improved by using a pressurizer such as those shown in U.S. Pat. Nos. 4,896,662 to Noble; 5,741,265 to Chan; and 6,017,350 to Long. Each of the pressurizers shown in these patents includes a body defining an inlet for receiving the nozzle of the bone cement injection device and an outlet in direct communication with the medullary canal. The body seals the medullary canal to pressurize the bone cement in the medullary canal. However, each of these pressurizers requires the nozzle to be positioned generally in line with the medullary canal to inject the bone cement into the medullary canal. Thus, these pressurizers require relatively straight-line access to the medullary canal.

### BRIEF SUMMARY OF THE INVENTION

The present invention provides a pressurizer for use with a bone cement injection device to inject bone cement into an anatomical site under pressure. The pressurizer includes a body having a proximal end and a distal end. The distal end is formed of a resilient material to seal against the anatomical site. An inlet is defined at the proximal end and an outlet is defined at the distal end. A channel interconnects the inlet and the outlet to convey the bone cement to the anatomical site. The channel defines a cement flow path that directionally varies between the inlet and the outlet such that the bone cement can be injected inline with the anatomical site while the bone cement injection device is positioned at an angle to the anatomical site.

One advantage of this configuration is a reduction in congestion of equipment near the anatomical site. With the configuration of the present invention, the bone cement injection device can be kept out of the way of the anatomical site, such as a medullary canal, during the procedure.

In another aspect of the present invention, the body includes a support member formed of rigid material and a wall of resilient material surrounds the rigid support member. The support member is preferably formed from a polycarbonate or like material and the wall is preferably formed from silicone.

In yet another aspect of the present invention, a handle is fixed to the body to manipulate the body during use, e.g., to facilitate placement of the body in the medullary canal. The handle may be fixed to the support member or to the wall or the handle may be a separate component.

### BRIEF DESCRIPTION OF THE SEVERAL VIEWS OF THE DRAWINGS

Advantages of the present invention will be readily appreciated as the same becomes better understood by reference to the following detailed description when considered in connection with the accompanying drawings wherein:

FIG. 1 is a perspective view of a pressurizer of the present invention being used with a bone cement injection device to inject bone cement into an anatomical site under pressure;

FIG. 2 is a cross-sectional perspective view of the pressurizer of FIG. 1 taken along the line 2-2 in FIG. 1;

FIG. 3 is an exploded view of an alternative embodiment of the pressurizer illustrating a support member and a wall of resilient material that is formed about the support member;

FIG. 4 is a perspective view of the pressurizer of the alternative embodiment of FIG. 4;

FIG. 5 is a perspective view of another alternative embodiment of the pressurizer in which a handle is fixed to a body of the pressurizer to facilitate manipulation of the body during use; and

FIG. 6 is a perspective view of another alternative embodiment of the pressurizer with the handle fixed to the support member of the body.

### DETAILED DESCRIPTION OF THE INVENTION

Referring to the Figures, wherein like numerals indicate like or corresponding parts throughout the several views, a pressurizer is generally shown at 14. The pressurizer 14 is preferably used in combination with a bone cement injection device 30 to form a bone cement injection system. The injection system provides for the injection of bone cement BC into an anatomical site 12, such as a bone canal 12, under pressure.

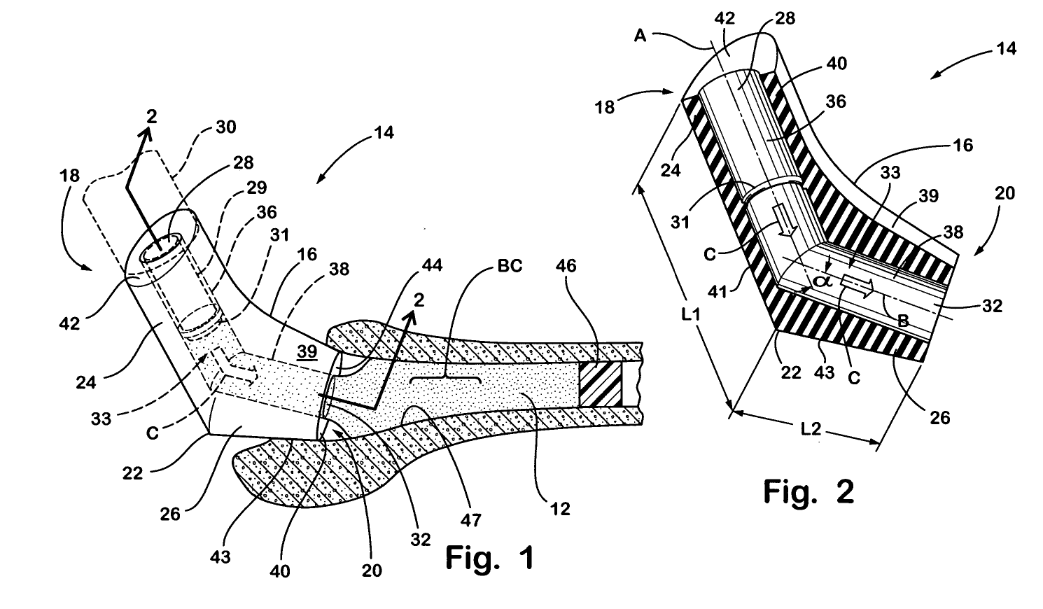

Referring to FIG. 1, the pressurizer 14 comprises a body 16 extending between a proximal end 18 and a distal end 20. The proximal end 18 of the body 16 is defined by a first planar surface 42 having a generally elliptical shape. The distal end 20 of the body 16 is defined by a second planar surface 44 having a generally elliptical shape. The planar surfaces 42, 44 are disposed in planes that are transverse to one another. The body 16 defines an inlet 28 in the first planar surface 42 and an outlet 32 in the second planar surface. The inlet 28 is adapted to receive a nozzle 29 of the bone cement injection device 30. The outlet 32 is adapted for operatively communicating with the bone canal 12. A generally circular channel 33 extends through the body 16 between the inlet 28 and the outlet 32.

Referring to FIG. 2, the channel 33 comprises first 36 and second 38 segments. The first segment 36 is disposed about a first axis A and extends from the inlet 28 to the second segment 38. The second segment 38 is disposed about a second axis B and extends from the first segment 36 to the outlet 32. The first A and second B axes intersect one another inside the body 16 at an acute angle α relative to one another. As a result, the first 36 and second 38 segments are arranged at the acute angle α to one another, i.e., are nonlinear relative to one another, such that the bone cement is redirected inside the body 16 as the bone cement travels from the first segment 36 into the second segment 38 during injection. In other words, still referring to FIG. 2, the channel 33 defines a cement flow path C that directionally varies between the inlet 28 and the outlet 32. The cement flow path C begins at an angle to the bone canal 12 in the first segment 36 and finishes in line with the bone canal 12 in the second segment 38. Preferably, the channel 33 maintains a substantially uniform diameter along the first 36 and second 38 segments between the inlet 28 and the outlet 32. The inlet 28 and outlet 32 and the first 36 and second 38 segments are preferably circular in shape.

The body 16 includes a wall 40 of resilient material, preferably elastomeric material, formed about the channel 33. The wall 40 is more preferably formed from silicone, and most preferably formed from silicone having a durometer of at least 40 Shore A. In alternative embodiments, the silicone may have an alternative stiffness or be replaced with a like material. As seen in the cross-sectional view of FIG. 2, the wall 40 varies in thickness between the proximal 18 and distal 20 ends. An annular seal 31 is integrally formed with the wall 40 in the first segment 36 of the channel 33 to seal the nozzle 29 in the first segment 36. In alternative embodiments, the annular seal 31 may be a separate o-ring that fits into a groove formed in the wall 40 in the first segment 36.

The wall 40 gives the body 16 a boot-shaped appearance with a front surface 39 extending in a curvilinear shape from the proximal end 18 to the distal end 20, a rear surface 41 extending from the proximal end 18 to a heel section 22, and a bottom surface 43 extending from the heel section 22 to the distal end 20.

Referring specifically to FIG. 2, the body 16 includes a first portion 24 lying generally along the first axis A from the proximal end 18 to the heel section 22 and a second portion 26 lying generally along the second axis B from the heel section 22 to the distal end 20. The first 24 and second 26 portions share the front surface 39. The rear surface 41 extends along the first portion 24 from the proximal end 18 to the heel section 22 and the bottom surface 43 extends along the second portion 26 from the heel section 22 to the distal end 20. The rear surface 41 has a length L1 that is from 20 to 100 percent longer than a length L2 of the bottom surface 43, more preferably, 30 to 70 percent longer than the length L2 of the bottom surface 43, and most preferably 30 to 50 percent longer than the length L2 of the bottom surface 43. Of course, in other embodiments, the lengths could vary outside of this range, or they could be equal. The first portion 24 gradually increases in size along the first axis A from the proximal end 18 to the heel section 22. Likewise, the second portion 26 gradually decreases in size along the second axis B from the heel section 22 to the distal end 20.

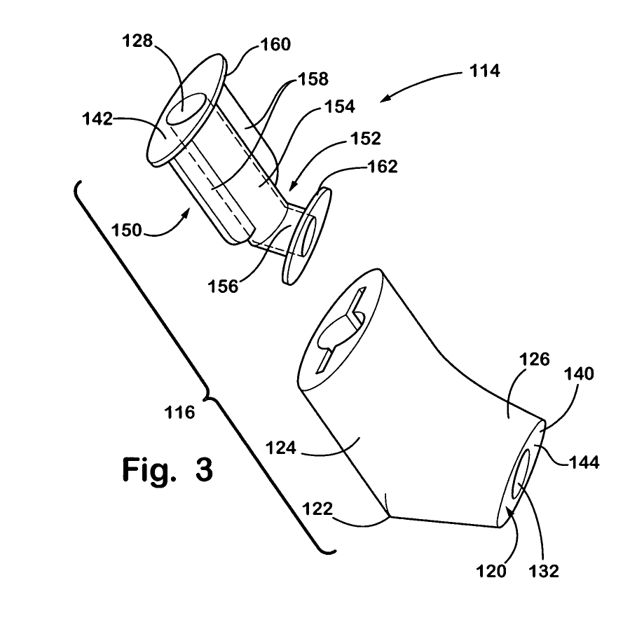

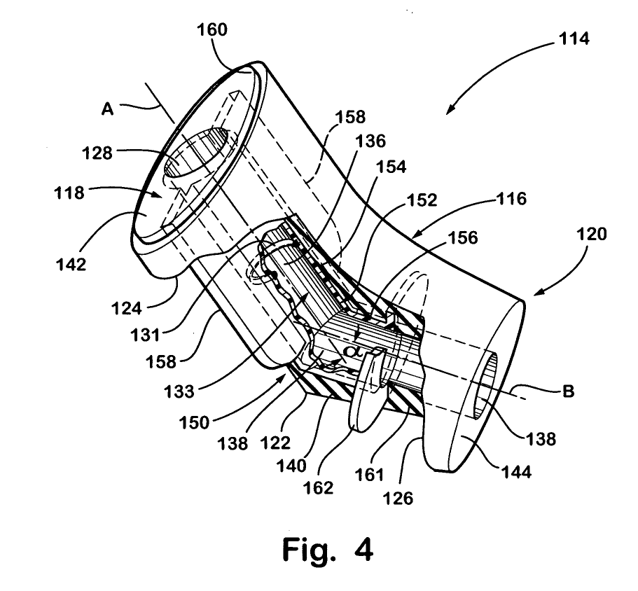

Referring to FIGS. 3 and 4, an alternative embodiment of the pressurizer 114 is shown. In this embodiment, the body 116 includes a rigid support member 150 to provide additional support and structural rigidity to the body 116 during use. This additional support is helpful in transferring additional axial pressure into the body 116 to help seal the pliable silicon wall 140 against the inner surface 47 of the bone canal 12. Hence, the rigid support member 150 further assists in transferring a force exerted by the user on the bone cement injection device 30 into the body 116 to increase the strength of the seal between the wall 140 and the inner surface 47. This further pressurizes the bone cement BC in the bone canal 12. FIG. 3 illustrates the rigid support member 150 separated out from the rest of the body 116. This is for illustrative purposes only. In practice, the wall 140 of resilient material is formed about the rigid support member 150. Preferably, the wall 140 is overmolded onto the resilient body 150.

Referring to FIG. 4, the rigid support member 150 includes a tubular member 152 extending between first and second ends to further define said channel 133. The tubular member 152 has a first tubular section 154 to further define the first segment 136 of the channel 133 and a second tubular section 156 angled at the acute angle α relative to the first first tubular section 154 to further define a portion of the second segment 138. A groove may be formed inside the first tubular section 154 to seat the annular seal 131. A first rigid flange 160 is fixed about the tubular member 152 and perpendicular to the tubular member 152 at the first end to further define the inlet 128 of the body 116.

Wings 158 are fixed on opposite sides of the tubular member 152 from the first rigid flange 160 distally along the first tubular section 154. The wings 158 extend radially outwardly from the first tubular section 154 into the wall 140 relative to the first axis A. The first rigid flange 160 perpendicularly intersects the wings 158. In this embodiment, the first rigid flange 160 presents the first planar surface 142 in which the inlet 128 is defined. A second rigid flange 162 is fixed about the tubular member 152 and perpendicular to the tubular member 152 at the second end.

The second rigid flange 162 is positioned within the body 116 between the heel section 122 and the distal end 120 of the body 116. Preferably, the second rigid flange 162 terminates distal of the heel section 122 and proximal to the distal end 120. Thus, the wall 140 of resilient material extends distally past the second end of the rigid support member 150 in an unsupported manner to define a flexible portion 161 for sealing against the inner surface 47 of the bone canal 12 as the bone cement BC is injected into the bone canal 12 through the pressurizer 114. Preferably, the wall 140 completely surrounds the rigid support member 150, except at the first flange 160. Thus, these two components are inseparable. The rigid support member 150 is preferably formed from a rigid material such as polycarbonate or like material, including stainless steel.

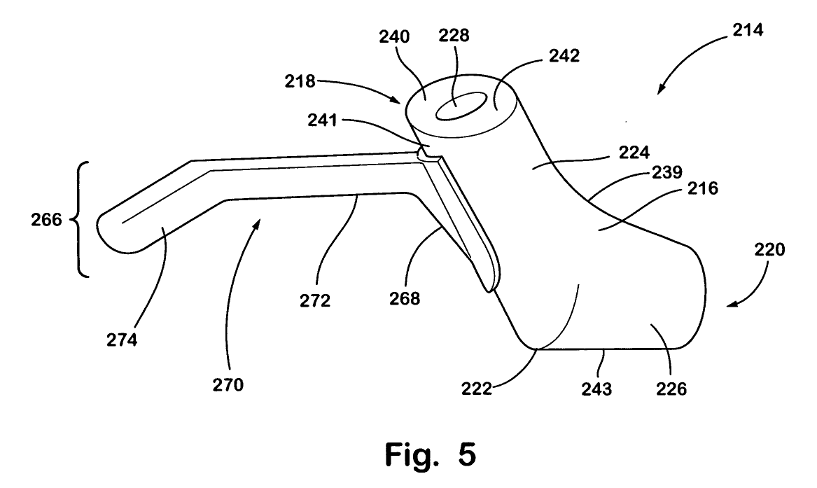

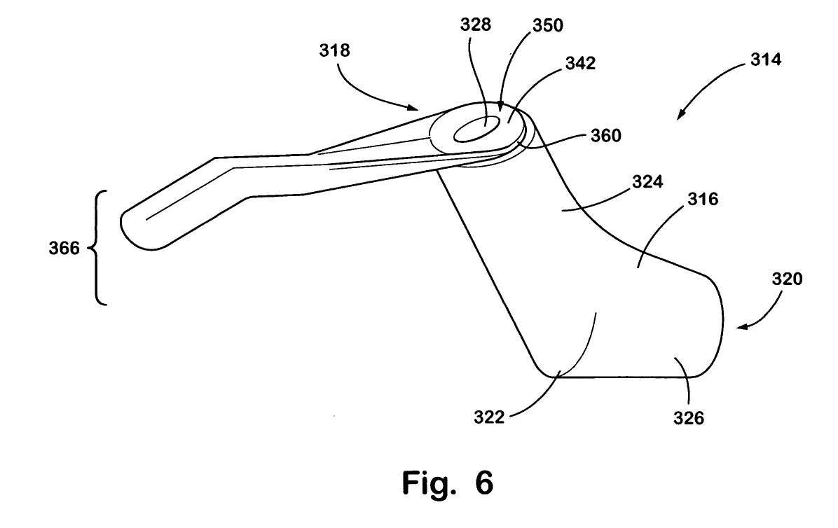

FIG. 5 illustrates yet another embodiment of the pressurizer 214. In this embodiment, a handle 266 is fixed to the body 216 to maneuver the body 216 during use. The handle 266 is preferably fixed to the rear surface 241 of the first portion 224. The handle 266 includes a base plate 268 having an arcuate shovel-like shape to match the shape of the rear surface 241 of the first portion 224. The base plate 268 is preferably mounted to the rear surface 241 of the first portion 224 by an adhesive, ultrasonic welding, interdigitation of prongs in the base plate 268, or by other methods well known in the art such that the base plate 268 is fixed to the body 216. An arm 270 extends from the base plate 268 in a cantilevered manner and includes a first section 272 and a second section 274 extending at an obtuse angle to the first section 272. The second section 274 lies generally in the same plane as the first planar surface 242. In a further embodiment 314 shown in FIG. 6, the handle 366 may be fixed to the rigid support member 350 at the first rigid flange 360. In other embodiments, the handle 266, 366 could also be a selectively removable component of the pressurizer 214, 314.

These handle arrangements provide additional mechanical advantage to the user when placing the body 216, 316 in the bone canal 12 and injecting the bone cement BC into the bone canal 12. This additional mechanical advantage can replace some of the force required from the user on the bone cement injection device 30 to ensure an adequate seal between the body 216 at the distal end 220, 320 and the inner surface 47 of the bone canal 12. Thus, the handle 266, 366 provides better maneuverability of the pressurizer 214, 314 and the bone cement injection device 30 in surgical procedures. In some instances in which the user can exert a majority of the required force using the handle 266, 366, a flexible nozzle (not shown) can replace the rigid nozzle 29, shown in FIG. 1.

During use, referring back to FIG. 1, the nozzle 29 of the bone cement injection device 30 is positioned within the first segment 36 and is sealed therein by the annular seal 31. The pressurizer 14, with nozzle 29 sealed thereto, is inserted into the bone canal 12. In particular, the distal end 20 is positioned within the bone canal 12 such that the front surface 39 and the bottom surface 43 adjacent to the distal end 20 creates a seal with an inner surface 47 of the bone canal 12. The distal end 20 is configured to provide a suitable sealing interface between the pressurizer 14 and the inner surface 47. This interface is critical in pressurizing the bone cement BC within the bone canal 12.

Once in sealed engagement, the bone cement BC is injected through the nozzle 30 into the first segment 36. The bone cement BC travels along the cement flow path C, where the bone cement BC is redirected in the second segment 38 toward the outlet 32 to be ultimately discharged from the outlet 32 into the bone canal 12. This configuration allows a user to inject the bone cement BC into the pressurizer 14 at an angle, while the bone cement BC is redirected to be discharged from the outlet 32 inline with the bone canal 12. Once the bone canal 12 is close to being filled, as shown in FIG. 1, the bone cement BC is injected under pressure. The pressurizer 14 maintains the seal within the bone canal 12 to facilitate pressurization of the bone cement BC during injection. The pressure in the bone canal 12 results from a combination of a force produced by the user on the bone cement injection device 30 and the seal of the pressurizer 14 within the bone canal 12. The pressure assists in reducing air pockets within the bone cement BC. The pressure also increases contact with cancellous bone and penetration into the cancellous bone along the inner surface 47 of the bone canal 12, thus increasing the structural integrity of the bone cement BC in THA or like procedures. A plug 46 is preferably used to seal a distal end of the bone canal 12 and prevent the distal migration of the bone cement BC during injection.

The embodiments described herein allow the surgeon in a THA or like procedure to inject the bone cement BC into the bone canal 12 at an angle to the bone canal 12. Often, this is necessary given the constraints of the anatomical site in which the procedure is being performed. Furthermore, these embodiments provide added flexibility and maneuverability in total hip arthroscopy and total knee arthroscopy procedures. These procedures are minimally invasive. The pressurizer 14, 114, 214, 314 of the present invention can be used in such procedures given its flexibility. For instance, in a muscle-sparing procedure, the maneuverability of the pressurizer 14, 114, 214, 314 and the bone cement injection device 30 allows the user to access the anatomical site at varying angles in between muscle layers. The pressurizer 14, 114, 214, 314 can similarly be used in a lateral approach to total knee arthroscopy.

Obviously, many modifications and variations of the present invention are possible in light of the above teachings.

1. A pressurizer for use with a bone cement injection device to inject bone cement into an anatomical site under pressure, comprising:
a body having an inlet for coupling with the bone cement injection device, an outlet for providing operative communication between the bone cement injection device and the anatomical site, and a channel defining a cement flow path between said inlet and said outlet;
said body having a proximal end and extending to a distal end formed of a resilient material for sealing with the anatomical site; and
said cement flow path directionally varying along said channel between said inlet and said outlet such that the bone cement can be injected inline with the anatomical site while the bone cement injection device is positioned at an angle to the anatomical site.

2. A pressurizer as set forth in claim 1 wherein said channel includes a first segment disposed about a first axis and a second segment disposed about a second axis forming an acute angle with said first axis.

3. A pressurizer as set forth in claim 2 wherein said first segment intersects said second segment and said cement flow path directionally varies between said first segment and said second segment.

4. A pressurizer as set forth in claim 3 wherein said first segment extends from said inlet to said intersection of said segments and said second segment extends from said intersection of said segments to said outlet.

5. A pressurizer as set forth in claim 2 wherein said body includes a seal protruding radially inward in said first segment relative to said first axis for sealing the bone cement injection device in said first segment.

6. A pressurizer as set forth in claim 2 wherein said body comprises a first portion lying generally along said first axis from said proximal end to a heel section and a second portion lying generally along said second axis from said heel section to said distal end.

7. A pressurizer as set forth in claim 6 wherein said first portion increases in size along said first axis between said proximal end and said heel section.

8. A pressurizer as set forth in claim 6 wherein said second portion decreases in size along said second axis between said heel section and said distal end.

9. A pressurizer as set forth in claim 6 wherein said first and second portions share a front surface that is curved from said proximal end to said distal end.

10. A pressurizer as set forth in claim 6 wherein said first portion has a rear surface extending from said proximal end to said heel section and said second portion has a bottom surface extending from said heel section to said distal end with said rear and bottom surfaces intersecting at said heel section.

11. A pressurizer as set forth in claim 10 wherein said rear surface is 30 to 50 percent longer than said bottom surface.

12. A pressurizer as set forth in claim 1 wherein said body is generally boot-shaped with a front surface extending in a curvilinear shape from said proximal end to said distal end, a rear surface extending from said proximal end to a heel section, and a bottom surface extending from said heel section to said distal end.

13. A pressurizer as set forth in claim 1 wherein said channel has a substantially uniform diameter.

14. A pressurizer as set forth in claim 1 wherein said body includes a wall of resilient material formed about said channel.

15. A pressurizer as set forth in claim 14 wherein said resilient material is an elastomeric material.

16. A pressurizer as set forth in claim 15 wherein said elastomeric material has a durometer of at least 40 Shore A.

17. A pressurizer as set forth in claim 1 including a handle coupled to said body for maneuvering said body while injecting the bone cement into the anatomical site.

18. A pressurizer as set forth in claim 17 wherein said body includes a rear surface and said handle includes a base fixed to said rear surface.

19. A pressurizer as set forth in claim 18 wherein said base is fixed to said rear surface using an adhesive, ultrasonic welding, or any combination thereof.

20. A pressurizer for use with a bone cement injection device to inject bone cement into an anatomical site under pressure, said pressurizer comprising:
a support member of rigid material;
a wall of resilient material formed about said support member to define a body having an inlet for coupling with the bone cement injection device, an outlet for providing operative communication between the bone cement injection device and the anatomical site, and a channel defining a cement flow path between said inlet and said outlet; and
said cement flow path directionally varying along said channel between said inlet and said outlet such that the bone cement can be injected inline with the anatomical site while the bone cement injection device is positioned at an angle to the anatomical site.

21. A pressurizer as set forth in claim 20 wherein said support member is substantially rigid and extends from a first end to a second end.

22. A pressurizer as set forth in claim 21 wherein said support member includes a tubular member extending from said first end to said second end to further define said channel of said body.

23. A pressurizer as set forth in claim 22 wherein said support member includes a first flange fixed about said tubular member at said first end to further define said inlet of said body.

24. A pressurizer as set forth in claim 22 wherein said support member includes a pair or planar wings fixed to said tubular member and extending in opposite directions from said tubular member into said wall of resilient material.

25. A pressurizer as set forth in claim 22 wherein said support member includes a second flange fixed about said tubular member at said second end.

26. A pressurizer as set forth in claim 21 wherein said wall of resilient material extends distally past said second end of said support member in an unsupported manner to define a flexible portion for sealing against the anatomical site as the bone cement is injected into the anatomical site through said pressurizer.

27. A pressurizer as set forth in claim 26 wherein said unsupported flexible portion further defines said channel between said support member and said distal end.

28. A pressurizer as set forth in claim 20 including a handle coupled to said body for maneuvering said body while injecting the bone cement into the anatomical site.

29. A pressurizer as set forth in claim 28 wherein said handle includes a base and said base is fixed to said support member at said first end.

30. A pressurizer for use with a bone cement injection device to inject bone cement into an anatomical site under pressure, said pressurizer comprising:
a body having an inlet for coupling with the bone cement injection device, an outlet for providing operative communication between the bone cement injection device and the anatomical site, and a channel defining a cement flow path between said inlet and said outlet; and
a handle coupled to said body for maneuvering said body independent of the bone cement injection device while injecting the bone cement into the anatomical site.

31. A pressurizer as set forth in claim 30 wherein said body includes a rear surface and said handle includes a base fixed to said rear surface.

32. A pressurizer as set forth in claim 31 wherein said base is fixed to said rear surface using an adhesive, ultrasonic welding, or any combination thereof.

33. A pressurizer as set forth in claim 30 wherein said body includes a support member of rigid material and a wall of resilient material formed about said support member and said base is fixed to said support member.

34. A pressurizer as set forth in claim 30 wherein said handle includes a base fixed to said body and an arm extending from said base in a cantilevered manner.

35. A bone cement injection system for injecting bone cement into an anatomical site, comprising:
a bone cement injection device;
a pressurizer comprising a body having an inlet for coupling with said bone cement injection device, an outlet for providing operative communication between said bone cement injection device and the anatomical site, and a channel defining a cement flow path between said inlet and said outlet;
said body having a proximal end and extending to a distal end formed of a resilient material for sealing with the anatomical site; and
said cement flow path directionally varying along said channel between said inlet and said outlet such that the bone cement can be injected inline with the anatomical site while said bone cement injection device is positioned at an angle to the anatomical site.

36. A bone cement injection system as set forth in claim 35 wherein said channel includes a first segment disposed about a first axis and a second segment disposed about a second axis forming an acute angle with said first axis.

37. A bone cement injection system as set forth in claim 36 wherein said first segment intersects said second segment at said acute angle and said cement flow path directionally varies between said first segment and said second segment.

38. A bone cement injection system as set forth in claim 37 wherein said first segment extends from said inlet to said intersection of said segments and said second segment extends from said intersection of said segments to said outlet.

39. A bone cement injection system as set forth in claim 36 wherein said body includes a seal engaging said bone cement injection device in said first segment.

40. A method of pressurizing bone cement injected into an anatomical site by a bone cement injection device using a pressurizer comprising a body having an inlet for coupling with the bone cement injection device, an outlet for providing operative communication between the bone cement injection device and the anatomical site, a channel defining a cement flow path between the inlet and the outlet, and a distal end formed of a resilient material for sealing with the anatomical site, said method comprising the steps of:
positioning the bone cement injection device into the inlet of the pressurizer;
sealing the distal end of the pressurizer with the anatomical site after positioning the bone cement injection device into the inlet with the bone cement injection device being positioned at an angle to the anatomical site;
injecting the bone cement through the channel, along the cement flow path, and into the anatomical site; and
varying a flow direction of the bone cement in the cement flow path while injecting the bone cement into the anatomical site such that the bone cement can be injected inline with the anatomical site while the bone cement injection device is positioned at the angle to the anatomical site.
